SEQUENCE LISTING (93 sequences)

SEQ ID NO: 1 - protein, 12 aa, Artificial
  synthetic peptide
XXXDXDXLIXXX

SEQ ID NO: 2 - protein, 6 aa, Artificial
  synthetic peptide
DEDTLI

SEQ ID NO: 3 - protein, 6 aa, Artificial
  synthetic peptide
DRDVLI

SEQ ID NO: 4 - protein, 10 aa, Artificial
  synthetic peptide
YGKKRRQRRR

SEQ ID NO: 5 - protein, 16 aa, Artificial
  synthetic peptide
CRQIKIWFQNRRMKWK

SEQ ID NO: 6 - protein, 13 aa, Artificial
  synthetic peptide
XXXVAXXXLXXTV

SEQ ID NO: 7 - protein, 7 aa, Artificial
  synthetic peptide
VARVELK

SEQ ID NO: 8 - protein, 7 aa, Artificial
  synthetic peptide
VADEKLH

SEQ ID NO: 9 - protein, 12 aa, Artificial
  synthetic peptide
XXXQXEXXAXXX

SEQ ID NO: 10 - protein, 6 aa, Artificial
  synthetic peptide
QVEQDA

SEQ ID NO: 11 - protein, 6 aa, Artificial
  synthetic peptide
QAEDEA

SEQ ID NO: 12 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 13 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 14 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 15 - protein, 673 aa, Homo sapiens
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPMKASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRTYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 16 - protein, 674 aa, Homo sapiens
MAPFLRISFNSYELGSLQVEDEASQPFCAVKMKEALSTERGKTLVQKKPTMYPEWKTTFDAHIYEGRVIQIVLMRAAEDPVSEVTVGVSVLAERCKKNNGKAEFWLDLQPQAKVLMCVQYFLEDGDCKQSMRSEEEAKFPTMNRRGAIKQAKIHYIKNHEFIATFFGQPTFCSVCKEFVWGLNKQGYKCRQCNAAIHKKCIDKIIGRCTGTATNSRDTIFQKERFNIDMPHRFKVYNYMSPTFCDHCGSLLWGLVKQGLKCEDCGMNVHHKCREKVANLCGINQKLLAEALNQVTQRSSRKLDTTESVGIYQGFEKKPEVSGSDILDNNGTYGKIWEGSTRCTLENFTFQKVLGKGSFGKVLLAELKGKDKYFAIKCLKKDVVLIDDDVECTMVEKRVLALAWESPFLTHLICTFQTKDHLFFVMEFLNGGDLMFHIQDKGRFELYRATFYAAEIICGLQFLHSKGIIYRDLKLDNVMLDRDGHIKIADFGMCKENIFGEGRASTFCGTPDYIAPEILQGLKYSFSVDWWSFGVLLYEMLIGQSPFHGDDEDELFESIRVDTPHYPRWITKESKDIMEKLFERDPDKRLGVTGNIRIHPFFKTINWSLLEKRKVEPPFKPKVKSPSDYSNFDPEFLNEKPQLSFSDKNLIDSMDQEAFHGFSFVNPKFEQFLDI

SEQ ID NO: 17 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 18 - protein, 160 aa, Homo sapiens
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPTMNRRGAIKQAKIHYIKNHE

SEQ ID NO: 19 - protein, 55 aa, Homo sapiens
PKVKSPSDYSNFDPEFLNEKPQLSFSDKNLIDSMDQEAFHGFSFVNPKFEQFLDI

SEQ ID NO: 20 - protein, 144 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEE

SEQ ID NO: 21 - protein, 735 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDRIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 22 - protein, 8 aa, Homo sapiens
MRAAEDPM

SEQ ID NO: 23 - protein, 8 aa, Homo sapiens
LQANRDPD

SEQ ID NO: 24 - protein, 13 aa, Homo sapiens
DEAQVEQDAQALF

SEQ ID NO: 25 - protein, 13 aa, Homo sapiens
GSLQAEDEANQPF

SEQ ID NO: 26 - protein, 6 aa, Homo sapiens
QVEADA

SEQ ID NO: 27 - protein, 12 aa, Homo sapiens
LGTDEDTLIEIL

SEQ ID NO: 28 - protein, 12 aa, Homo sapiens
AHIDRDVLIVLV

SEQ ID NO: 29 - protein, 6 aa, Homo sapiens
DADVLI

SEQ ID NO: 30 - protein, 16 aa, Homo sapiens
LSAVARVELKGTVRPA

SEQ ID NO: 31 - protein, 21 aa, Homo sapiens
KAEVADEKLHVTVRDAKNLIP

SEQ ID NO: 32 - protein, 7 aa, Homo sapiens
VAAVALK

SEQ ID NO: 33 - protein, 21 aa, Homo sapiens
YGRKKRRQRRRCCLQANEDPD

SEQ ID NO: 34 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 35 - protein, 23 aa, Homo sapiens
RKLMVALAKGRRAEDGSVIDYEL

SEQ ID NO: 36 - protein, 22 aa, Homo sapiens
RKALLTLADGRRDESLKVDEHL

SEQ ID NO: 37 - protein, 22 aa, Homo sapiens
QRVLVSLSAGGRDEGNYLDDAL

SEQ ID NO: 38 - protein, 22 aa, Homo sapiens
ERLLVSMCQGNRDENQSINHQM

SEQ ID NO: 39 - protein, 23 aa, Homo sapiens
ERILVCLLQGSRDDVSSFVDPAL

SEQ ID NO: 40 - protein, 23 aa, Homo sapiens
QDLLLALAKGGRDSYSGIIDYNL

SEQ ID NO: 41 - protein, 21 aa, Homo sapiens
RDTLMNLVQGTREEGYTDPAM

SEQ ID NO: 42 - protein, 22 aa, Homo sapiens
QRLLISLSQGNRDESTNVDMSL

SEQ ID NO: 43 - protein, 22 aa, Homo sapiens
KKILVSLLQANRNEGDDVDKDL

SEQ ID NO: 44 - protein, 24 aa, Homo sapiens
ERLLVSLVQANRDRVGKVDEGQVE

SEQ ID NO: 45 - protein, 24 aa, Homo sapiens
QRLLVVLLQANRDRDGRVDEALVE

SEQ ID NO: 46 - protein, 24 aa, Homo sapiens
QRMLVVLLQANRDRDTAIDDAQVE

SEQ ID NO: 47 - protein, 24 aa, Homo sapiens
QRMLVVLLQANRDRDAGIDEAQVE

SEQ ID NO: 48 - protein, 24 aa, Homo sapiens
QRMLVVLLQANRDRDARIDEAQVE

SEQ ID NO: 49 - protein, 11 aa, Artificial
  synthetic peptide
XXDXDXLIXXX

SEQ ID NO: 50 - protein, 11 aa, Artificial
  synthetic peptide
XXXDXDXLIXX

SEQ ID NO: 51 - protein, 9 aa, Artificial
  synthetic peptide
XXDXDXLIX

SEQ ID NO: 52 - protein, 9 aa, Artificial
  synthetic peptide
XDXDXLIXX

SEQ ID NO: 53 - protein, 8 aa, Artificial
  synthetic peptide
XDXDXLIX

SEQ ID NO: 54 - protein, 10 aa, Artificial
  synthetic peptide
XXXDXDXLIX

SEQ ID NO: 55 - protein, 9 aa, Artificial
  synthetic peptide
XXXDXDXLI

SEQ ID NO: 56 - protein, 8 aa, Artificial
  synthetic peptide
XXDXDXLI

SEQ ID NO: 57 - protein, 7 aa, Artificial
  synthetic peptide
XDXDXLI

SEQ ID NO: 58 - protein, 9 aa, Artificial
  synthetic peptide
DXDXLIXXX

SEQ ID NO: 59 - protein, 10 aa, Artificial
  synthetic peptide
XDXDXLIXXX

SEQ ID NO: 60 - protein, 8 aa, Artificial
  synthetic peptide
DXDXLIXX

SEQ ID NO: 61 - protein, 7 aa, Artificial
  synthetic peptide
DXDXLIX

SEQ ID NO: 62 - protein, 6 aa, Artificial
  synthetic peptide
DXDXLI

SEQ ID NO: 63 - protein, 12 aa, Artificial
  synthetic peptide
XXVAXXXLXXTV

SEQ ID NO: 64 - protein, 11 aa, Artificial
  synthetic peptide
XXXVAXXXLXX

SEQ ID NO: 65 - protein, 10 aa, Artificial
  synthetic peptide
XXXVAXXXLX

SEQ ID NO: 66 - protein, 12 aa, Artificial
  synthetic peptide
XXXVAXXXLXXT

SEQ ID NO: 67 - protein, 9 aa, Artificial
  synthetic peptide
XXVAXXXLX

SEQ ID NO: 68 - protein, 11 aa, Artificial
  synthetic peptide
XXVAXXXLXXT

SEQ ID NO: 69 - protein, 10 aa, Artificial
  synthetic peptide
XXVAXXXLXX

SEQ ID NO: 70 - protein, 9 aa, Artificial
  synthetic peptide
XVAXXXLXX

SEQ ID NO: 71 - protein, 8 aa, Artificial
  synthetic peptide
XVAXXXLX

SEQ ID NO: 72 - protein, 7 aa, Artificial
  synthetic peptide
VAXXXLX

SEQ ID NO: 73 - protein, 11 aa, Artificial
  synthetic peptide
XVAXXXLXXTV

SEQ ID NO: 74 - protein, 10 aa, Artificial
  synthetic peptide
VAXXXLXXTV

SEQ ID NO: 75 - protein, 10 aa, Artificial
  synthetic peptide
XVAXXXLXXT

SEQ ID NO: 76 - protein, 9 aa, Artificial
  synthetic peptide
VAXXXLXXT

SEQ ID NO: 77 - protein, 11 aa, Artificial
  synthetic peptide
XXXQXEXXAXX

SEQ ID NO: 78 - protein, 10 aa, Artificial
  synthetic peptide
XXXQXEXXAX

SEQ ID NO: 79 - protein, 9 aa, Artificial
  synthetic peptide
XXXQXEXXA

SEQ ID NO: 80 - protein, 11 aa, Artificial
  synthetic peptide
XXQXEXXAXXX

SEQ ID NO: 81 - protein, 10 aa, Artificial
  synthetic peptide
XXQXEXXAXX

SEQ ID NO: 82 - protein, 9 aa, Artificial
  synthetic peptide
XXQXEXXAX

SEQ ID NO: 83 - protein, 8 aa, Artificial
  synthetic peptide
XXQXEXXA

SEQ ID NO: 84 - protein, 10 aa, Artificial
  synthetic peptide
XQXEXXAXXX

SEQ ID NO: 85 - protein, 9 aa, Artificial
  synthetic peptide
XQXEXXAXX

SEQ ID NO: 86 - protein, 8 aa, Artificial
  synthetic peptide
XQXEXXAX

SEQ ID NO: 87 - protein, 7 aa, Artificial
  synthetic peptide
XQXEXXA

SEQ ID NO: 88 - protein, 6 aa, Artificial
  synthetic peptide
QXEXXA

SEQ ID NO: 89 - protein, 9 aa, Artificial
  synthetic peptide
QXEXXAXXX

SEQ ID NO: 90 - protein, 8 aa, Artificial
  synthetic peptide
QXEXXAXX

SEQ ID NO: 91 - protein, 7 aa, Artificial
  synthetic peptide
QXEXXAX

SEQ ID NO: 92 - protein, 10 aa, Artificial
  synthetic peptide
XXDXDXLIXX

SEQ ID NO: 93 - protein, 8 aa, Artificial
  synthetic peptide
VAXXXLXX

(19) United States
(12) Patent Application Publication
(10) Pub. No.: US 2009/0081786 A1
(43) Pub. Date: Mar. 26, 2009

(54) Peptide sequences for modulation of protein kinase C

(76) Inventors: Viktoria Kheifets, East Palo Alto, CA (US); Daria D. Mochly-Rosen, Menlo Park, CA (US)

Correspondence Address: King & Spalding LLP, P.O. Box 889, Belmont, CA, 94002-0889, US

(21) Appl. No.: 11/824,955

(22) Filed: Jul. 2, 2007

Related U.S. Application Data
(60) Provisional application No. 60/817,761, filed on Jun. 30, 2006.

(51) Int. Cl. C12N 5/06; C07K 7/00

(52) U.S. Cl. 435/375; 530/330; 530/329; 530/328; 530/326

(57) ABSTRACT

Peptides found within an annexin protein can be used to modulate the activity of a protein kinase C are described. More particularly, peptides within annexin I, V, and VI, as well as variants and conservatively modified variants thereof, are described, and use of these peptides to regulate cellular responses mediated by β-protein kinase C, δ-protein kinase C, and α-protein kinase C, respectively, are described.

### CROSS REFERENCE

This application claims the benefit of U.S. Provisional Patent Application No. 60/817,761, filed Jun. 30, 2006, which is hereby incorporated herein in its entirety by reference.

### TECHNICAL FIELD

The teachings provided herein relate to peptides found within an annexin that can be used to modulate the activity of a protein kinase C. More particularly, the teachings are directed to such peptides within annexin I, V, and VI, as well as variants and conservatively modified variants thereof, and to the use of these sequences to regulate cellular responses mediated by β-protein kinase C, δ-protein kinase C, and α-protein kinase C, respectively.

### BACKGROUND

Many biological processes involve specific protein-protein interactions. Protein-protein interactions, which may be transient or otherwise, enable two or more proteins or subunits to associate. Protein-protein interactions can have a number of measurable effects in that they can: alter kinetic properties of proteins; be a common mechanism for allowing substrate channeling; result in the formation of new binding sites; alter the activity of a protein; and/or alter the specificity of a protein for its substrate (Phizicky and Fields, Microbiological Reviews, 59:94-123, (1995); Pawson and Nash, Gene Dev., 14:1027-1047, (2000)).

The protein kinase C(PKC) family consists of several lipid-activated isozymes playing key roles in many signal transduction pathways. Three groups of PKC have been distinguished:

(1) the conventional, calcium-, and phospholipid-, and diacylglycerol (DAG)-dependent isoenzymes alpha (α), beta (β), and gamma (γ);

(2) the novel forms, delta (δ), epsilon (ε), theta (θ), and eta (η), which are calcium-independent; and,

(3) the atypical isoenzymes zeta (ζ) and iota (ι) which are both calcium- and DAG-independent.

These isoenzymes exhibit different tissue distributions and activator requirements, and each have their own individual roles in intracellular signaling. Activation of PKCs often involves translocation from the cytosolic fraction of a cell to the particulate or membrane compartments, or between different intracellular locations, and when a number of isozymes are translocated upon stimulation of the cell, different redistributions have been observed. This suggests that there are isozyme-specific interactions with membrane and cytoskeletal proteins both before and upon PKC activation, and such divergence may enable phosphorylation of different proteins that are already compartmentalized at the various sites.

A number of proteins which interact with PKCs have been reported. For example, inactive PKC may be localized by scaffolding proteins such as AKAP 79, and released upon lipid hydrolysis to phosphorylate co-localized proteins (Klauck, T. et al., Science, 271:1589 (1996)). Scaffolding proteins may also co-ordinate the actions of other kinases and phosphatases to promote cross-talk and signal termination. Proteins which bind PKC in a phospholipid-dependent manner have been described, such as receptors for activated C-kinase (RACKs) (Mochly-Rosen, D., Science, 268:247 (1995)) and cytoskeletal proteins such as vinculin and talin (Jaken, S. Curr. Opin Cell Biol., 8:168 (1996)). Some RACKs are isozyme specific and use of RACK-derived peptides to block individual isozyme relocalization interferes with specific cell functions (Yedovitzky, M. et al., J. Biol. Chem., 272:1417 (1997); Johnson, J. A. et al., J. Biol. Chem., 271:24962 (1996)).

Annexins are a family of structurally-related proteins found in diverse eukaroytic organisms such as the fruit fly, sponges, slime molds, higher plants, and mammals (Towle, C. A. et al., J. Biol. Chem., 267:5416 (1992)). Proteins in this family reversibly bind to negatively charged phospholipids (phosphatidylcholine and phosphatidylserine) in a calcium dependent manner. Many of the functions attributed to annexins are believed to be the result of this binding property. These functions include (1) regulation of phospholipase A2 activity, (2) anticoagulant activity, (3) roles in cellular exocytosis, (4) membrane trafficking, (5) cytoskeletal organization, (6) phosphohydrolase activity, (7) various aspects of cell proliferation, and (8) calcium channel activity (Towle et al., Id.). Annexin V, for example, is a specific family member found in a variety of species including human. It is widely distributed in various cells and tissues and is particularly abundant in brain, where it is believed to act as a paracrine-type neurotrophic factor (Ohsawa, K. et al. J. Neurochem., 67:89 (1996)). It is also known to possess anticoagulant activity, transport Ca²⁺ ions across phospholipid membranes, and inhibit phospholipase A2.

To date, there is no functional link established between the interactions of the annexins and the PKCs. There are, however, some connections that have been recognized between the PKCs and diseases. There remains a need in the art, however, for therapeutic agents capable of regulating the activity of the PKCs such as, for example, for regulating the activity of δ-PKC in its role associated with ischemia and reperfusion injury, for regulating α-PKC in its role associated with cancer, and for regulating β-PKC in its role associated with cardiac hypertrophy.

The foregoing examples of the related art and limitations related therewith are intended to be illustrative and not exclusive. Other limitations of the related art will become apparent to those of skill in the art upon a reading of the specification and a study of the drawings.

### SUMMARY

The present teachings are generally directed to amino acid sequences within the annexin family, such as annexin I, V, and VI, as well as variants and conservatively modified variants thereof, and the use of these sequences to regulate cellular responses mediated by β-protein kinase C, δ-protein kinase C, and α-protein kinase C, respectively.

In some embodiments, the teachings include a peptide comprising the amino acid sequence J_{N}-DXₐₐ₁DXₐₐ₂LI-J_{C} (SEQ ID NO:1), or a conservatively modified variant thereof, wherein Xₐₐ₁ is selected from a group consisting of E, R, A, and conservative substitutions thereof; Xₐₐ₂ is selected from a group consisting of T, V, and conservative substitutions thereof; J_{N} comprises a component selected from a group consisting of hydrogen; poly(ethylene glycol); methyl; T, GT, LGT, I, HI, AHI, and conservative substitutions thereof; and combinations thereof; and, J_{C} comprises a component selected from a group consisting of hydroxyl; poly(ethylene glycol); amino; and E, EI, EIL, V, VL, VLV, and conservative substitutions thereof; and combinations thereof.

In some embodiments, the peptide comprises DEDTLI (SEQ ID NO:2) or a conservatively modified variant thereof. In some embodiments, the peptide comprises DRDVLI (SEQ ID NO:3) or a conservatively modified variant thereof. In some embodiments, the peptide is further conjugated to a carrier peptide, and the carrier peptide can include TAT (SEQ ID NO:4), Drosophila Antennapedia homeodomain (SEQ ID NO:5), or a polyarginine.

In some embodiments, the teaching includes a peptide comprising the amino acid sequence J_{N}-VAXₐₐ₁Xₐₐ₂Xₐₐ₃ LXₐₐ₄-J_{C} (SEQ ID NO:6), or a conservatively modified variant thereof, wherein Xₐₐ₁ is selected from a group consisting of R, D, A, and conservative substitutions thereof; Xₐₐ₂ is selected from a group consisting of V, E, and conservative substitutions thereof; Xₐₐ₃ is selected from a group consisting of E, K, A, and conservative substitutions thereof; Xₐₐ₄ is selected from a group consisting of K, H, and conservative substitutions thereof; J_{N} comprises a component selected from a group consisting of hydrogen; poly(ethylene glycol); methyl; A, SA, LSA, E, AE, KAE, and conservative substitutions thereof; and combinations thereof; and, J_{C} comprises a component selected from a group consisting of hydroxyl; poly(ethylene glycol); amino; and G, GT, GTV, V, VT, VTV, and conservative substitutions thereof; and combinations thereof.

In some embodiments, the peptide comprises VARVELK (SEQ ID NO:7) or a conservatively modified variant thereof. In some embodiments, the peptide comprises VADEKLH (SEQ ID NO:8) or a conservatively modified variant thereof. In some embodiments, the peptide is further conjugated to a carrier peptide, wherein the carrier peptide can include TAT (SEQ ID NO:4), Drosophila Antennapedia homeodomain (SEQ ID NO:5), or polyarginine.

In some embodiments, the teaching includes a peptide comprising the amino acid sequence J_{N}-QXₐₐ₁EXₐₐ₂Xₐₐ₃A-J_{C} (SEQ ID NO:9), or a conservatively modified variant thereof, wherein Xₐₐ₁ is selected from a group consisting of V, A, and conservative substitutions thereof; Xₐₐ₂ is selected from a group consisting of Q, D, A, and conservative substitutions thereof; Xₐₐ₃ is selected from a group consisting of D, E, and conservative substitutions thereof; J_{N} comprises a component selected from a group consisting of hydrogen; poly(ethylene glycol); methyl; A, EA, DEA, L, SL, GSL, and conservative substitutions thereof; and combinations thereof; and, J_{C} comprises a component selected from a group consisting of hydroxyl; poly(ethylene glycol); amino; and Q, QA, QAL, N, NQ, NQP, and conservative substitutions thereof; and combinations thereof.

In some embodiments, the peptide comprises QVEQDA (SEQ ID NO:10) or a conservatively modified variant thereof. In some embodiments, the peptide comprises QAEDEA (SEQ ID NO:11) or a conservatively modified variant thereof. In some embodiments, the peptide is further conjugated to a carrier peptide, wherein the carrier peptide can include TAT (SEQ ID NO:4), Drosophila Antennapedia homeodomain (SEQ ID NO:5), or polyarginine.

In some embodiments, the teaching includes a method of increasing or decreasing a cellular response to β-protein kinase C (β-PKC) (βI is SEQ ID NO:12; βII is SEQ ID NO:34), wherein the method comprises adding a peptide from annexin I to an environment comprising a cell, annexin I (SEQ ID NO:13), and β-PKC; wherein, the peptide binds to the β-PKC and inhibits binding between the β-PKC and the annexin I. In some embodiments, the cellular response includes translocation of the β-PKC from the cytosolic fraction of cell to the particulate fraction of the cell. In some embodiments, the cellular response includes cellular hypertrophy. In some embodiments, the cellular response includes hyperproliferation.

In some embodiments, the teaching includes a method of increasing or decreasing a cellular response to α-protein kinase C (α-PKC) (SEQ ID NO:14), wherein the method comprises adding a peptide from annexin VI (SEQ ID NO:15) to an environment comprising a cell, annexin VI, and α-PKC; wherein, the peptide binds to the α-PKC and inhibits binding between the α-PKC and the annexin VI. In some embodiments, the cellular response includes translocation of the α-PKC from the cytosolic fraction of cell to the particulate fraction of the cell. In some embodiments, the cellular response includes hyperproliferation.

In some embodiments, the teaching includes a method of increasing or decreasing a cellular response to δ-protein kinase C (δ-PKC) (SEQ ID NO:16), wherein the method comprises adding a peptide from annexin V (SEQ ID NO:17) to an environment comprising a cell, annexin V, and δ-PKC; wherein, the peptide binds to the δ-PKC and inhibits binding between the δ-PKC and the annexin V. In some embodiments, the cellular response includes translocation of the δ-PKC from the cytosolic fraction of cell to the particulate fraction of the cell. In some embodiments, the cellular response includes cardioprotection following an ischemia/reperfusion injury.

### BRIEF DESCRIPTION OF THE FIGURES

FIGS. 1 A-D show that the domains V1/C2 and V5 of δ-PKC, as well as full-length δ-PKC, were observed to bind to annexin V, according to some embodiments.

FIG. 2 shows the effect of stimulation on the formation of a complex between δ-PKC and annexin V, according to some embodiments.

FIG. 3 shows the timing of the binding of δ-PKC to annexin V relative to translocation of the δ-PKC in a cell, according to some embodiments.

FIGS. 4A and 4B show the results of a real-time fluorescent imaging of the interaction of δ-PKC and annexin V in a cell, according to some embodiments.

FIGS. 5A-D show the effect of a peptide designed to inhibit the translocation of δ-PKC, according to some embodiments.

FIGS. 6 A-D show that association of δ-PKC with annexin V is needed for downstream signaling in an ex vivo model of ischemia and reperfusion, according to some embodiments.

### DETAILED DESCRIPTION

### I. Definitions

It must be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art. The protein sequences, for example, are presented herein using the one-letter or three-letter amino acid symbols that are commonly used in the art and are used in accordance with the recommendations of the IUPAC-IUB Biochemical Nomenclature Commission.

Any methods and materials similar or equivalent to those described herein can be used in the practice or testing of the teachings herein. All publications mentioned herein are incorporated herein by reference for the purpose of describing and disclosing the methodologies which are reported in the publications which might be used in connection with the teachings herein.

The term “substantially purified”, as used herein, refers to nucleic or amino acid sequences that are removed from their natural environment, isolated or separated, and are at least 60% free, preferably 75% free, more preferably 90% free, and most preferably 95% free from other components with which they are naturally associated.

“Peptide” and “polypeptide” are used interchangeably herein and refer to a compound made up of a chain of amino acid residues linked by peptide bonds. Unless otherwise indicated, the sequence for peptides is given in the order from the amino terminus to the carboxyl terminus.

The term “variant” refers to modifications to a peptide that allows the peptide to retain its binding properties, and such modifications include, but are not limited to, conservative substitutions in which one or more amino acids are substituted for other amino acids; deletion or addition of amino acids that have minimal influence on the binding properties or secondary structure; conjugation of a linker; post-translational modifications such as, for example, the addition of functional groups.

A “conservatively modified variant” is a variation that results in a conservative amino acid substitution. A “conservative amino acid substitution” is an amino acid substituted by an alternative amino acid of similar charge density, hydrophilicity/hydrophobicity, size, and/or configuration (e.g., Val for IIe). In comparison, a “nonconservatively modified variant” is a variation on the nucleic acid sequence that results in a nonconservative amino acid substitution. A “nonconservative amino acid substitution” is an amino acid substituted by an alternative amino acid of differing charge density, hydrophilicity/hydrophobicity, size, and/or configuration (e.g., Val for Phe). The means of making such modifications are well known in the art.

A “substitution”, as used herein, refers to the replacement of one or more amino acids by different amino acids, respectively. An “insertion” or “addition”, as used herein, refers to a change in an amino acid sequence resulting in the addition of one or more amino acid residues, as compared to the naturally occurring molecule. A “deletion”, as used herein, refers to a change in the amino acid sequence and results in the absence of one or more amino acid residues. A “modification” of an amino acid sequence or a “modified” amino acid sequence refers to an amino acid sequence that results from the addition of one or more amino acid residues, to either the N-terminus or the C-terminus of the sequence.

The term “modulate”, as used herein, refers to a change in the activity of a protein kinase C. For example, a modulation may cause an increase or a decrease in protein activity, binding characteristics, or any other biological, functional or immunological properties of a PKC.

The peptides taught herein typically show substantial sequence identity to a corresponding native peptide sequence. The term “substantial sequence identity” means that the two subject amino acid sequences, when optimally aligned, such as by the programs GAP or BESTFIT using default gap penalties, share at least 65 percent sequence identity, commonly 80-85% percent sequence identity, often at least 90-95 percent or greater sequence identity. “Percentage amino acid identity” refers to a comparison of the amino acid sequences of two peptides which, when optimally aligned, have approximately the designated percentage of the same amino acids. Sequence comparisons are generally made to a reference sequence over a comparison window of at least 5 residue positions, frequently over a window of at least 10-20 amino acids, wherein the percentage of sequence identity is calculated by comparing a reference sequence to a second sequence, the latter of which may represent, for example, a peptide analog sequence that includes one or more deletions, substitutions or additions which total 20 percent, typically less than 5-10% of the reference sequence over the window of comparison. The reference sequence may be a subset of a larger sequence, for example, a subset of residues from a protein taught herein. Optimal alignment of sequences for aligning a comparison window may be conducted according to the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), or by computerized implementations of these algorithms (GAP, BESTFIT, FASTA, and/or TFASTA, e.g., as provided in the Wisconsin Genetics Software Package Release 7.0, Genetics Computer Group, 575 Science Dr., Madison, Wis.).

The terms “identity,” “identical,” “homology,” and “homologous,” may be used synonymously. In some embodiments, the differences that affect the identity between two sequences can include, for example, a modification, such as a deletion or insertion within a sequence. It should be appreciated to one of skill that the polypeptides, in some embodiments, can be produced from two non-homologous polynucleotide sequences within the limits of degeneracy.

In some embodiments, the sequences taught herein can have substantial sequence identity and, thus, be at least 80, 85, 90, or 95 percent identical to a particular sequence having a known functionality, wherein the general functionality of the sequence is preserved, such that the sequence may have the same, less, or more activity than the particular sequence taught herein. As such, in some embodiments, the polypeptides taught herein can be at least 80, 85, 90, 95 or 97 percent identical to the peptides taught and described herein and, in these embodiments, the peptides can share the same general functionality.

In some embodiments, the general functionality of a polypeptide taught herein can be the regulation of a PKC mediated cellular activity. In some embodiments, a cellular activity can be increased, decreased, or maintained at a desired level. Examples of cellular activities include, but are not limited to, translocation of beta-PKC from the cytosolic fraction of a cell to the particulate fraction of the cell; cellular hypertrophy; hyperproliferation; and reperfusion following ischemia.

### II. Peptide and Peptide Compositions

The teachings are generally directed to amino acid sequences within the annexin family, such as annexin I (SEQ ID NO:13), annexin V (SEQ ID NO:17), and annexin VI (SEQ ID NO:15), as well as variants and conservatively modified variants thereof, and the use of these sequences to regulate cellular responses mediated by β-protein kinase C (βI is SEQ ID NO:12; βII is SEQ ID NO:34), δ-protein kinase C (SEQ ID NO:16), and α-protein kinase C (SEQ ID NO:14), respectively. The teaching is based on the identification of a peptide sequence within an annexin family that is capable of binding to a PKC, and thereby capable of regulating a PKC mediated cellular activity.

The annexin peptides that have been identified in the teachings as an agonist or antagonist of their respective PKC are unique not only to that PKC but also within the annexin protein family. For example, an annexin I peptide sequence that has an identity to a β-PKC cannot be found in any of the other annexin species. However, the annexin I peptide sequence can be found in any species of animal having the protein, as the sequences are conserved among the different species of animals. Therefore, a desired annexin peptide can be found in any of the species carrying that annexin including rats, humans, mice, cows, and fish. This suggests that sequences described herein are important to the specific interactions occurring between the annexins and their respective PKCs, regardless of the species of animal in which the interactions are occurring.

There are at least three examples of peptides predicted from identity alignments with a similar pattern that have been shown to be useful in the modulation of a PKC: β-PKC/RACK1, ε-PKC/RACK2, and δ-PKC/annexin V. These peptides follow the same principle: peptides designed from the PKC partner protein is an inhibitor of PKC function, whereas peptides designed from the PKC is an activator of PKC. Interactions between β-PKC/annexin I and α-PKC/annexin VI are recognized in the art. In fact, an independent peptide designed to block a β-PKC/annexin I interaction indeed blocked β-PKC function in cells. Annexins have been shown to be involved in the translocation processes of PKCs, for example, annexin V is involved in translocation process of δ-PKC. There are 11 annexin family members and 11 PKC family members. Homology sequences between different pairs of annexins and PKCs are indicative of a common role of annexins in PKC translocation.

Sequence homology is located on the same surface of a PKC for the two classical isozymes alpha and beta, suggesting that this site could be participating in important protein-protein interaction. This site in ε-PKC is involved in calponin binding (Leinweber JBC 2000 Dec. 22; 275(51):40329-36), again suggesting that this site is important to protein-protein interactions.

### Peptides Developed to Control β-PKC/Annexin I Interactions

A link between annexin I and beta PKC has been reported (Ron JBC 1994 Aug. 26; 269(34):21395-8). The peptides described herein were identified through a homology search between annexin I (SEQ ID NO:13) and β-PKC (βI is SEQ ID NO:12; βII is SEQ ID NO:34). The sequences are highly homologous except for a charge difference indicative of a potential protein-protein interaction similar to the interactions between β-IIPKC and RACK1, ε-PKC and RACK2, and δ-PKC and annexin V.

In some embodiments, the teaching includes a peptide comprising the amino acid sequence J_{N}-DXₐₐ₁DXₐₐ₂LI-J_{C} (SEQ ID NO:1), or a conservatively modified variant thereof, wherein Xₐₐ₁ is selected from a group consisting of E, R, A, and conservative substitutions thereof; Xₐₐ₂ is selected from a group consisting of T, V, and conservative substitutions thereof; J_{N} comprises a component selected from a group consisting of hydrogen; poly(ethylene glycol); methyl; T, GT, LGT, I, HI, AHI, and conservative substitutions thereof; and combinations thereof; and, J_{C} comprises a component selected from a group consisting of hydroxyl; poly(ethylene glycol); amino; and E, EI, EIL, V, VL, VLV, and conservative substitutions thereof; and combinations thereof.

In some embodiments, the peptide comprises DEDTLI (SEQ ID NO:2) or a conservatively modified variant thereof. In some embodiments, the peptide comprises DRDVLI (SEQ ID NO:3) or a conservatively modified variant thereof. In some embodiments, the peptide is further conjugated to a carrier peptide.

### Peptides Developed to Control α-PKC/Annexin VI Interactions

A link between α-PKC and annexin VI from skeletal muscle has been reported (Schmitz-Peiffer, C., et al., Biochem. J., 330:675 (1998)). The peptides taught herein have been identified through a homology search between annexin VI (SEQ ID NO:15) and α-PKC (SEQ ID NO:14). The sequences are highly homologous except for a charge difference indicative of a potential protein-protein interaction similar to the interactions between β-II PKC and RACK1, ε-PKC and RACK2, and δ-PKC and annexin V.

In some embodiments, the teaching includes a peptide comprising the amino acid sequence J_{N}-VAXₐₐ₁Xₐₐ₂Xₐₐ₃LXₐₐ₄-J_{C} (SEQ ID NO:6), or a conservatively modified variant thereof, wherein Xₐₐ₁ is selected from a group consisting of R, D, A, and conservative substitutions thereof; Xₐₐ₂ is selected from a group consisting of V, E, and conservative substitutions thereof; Xₐₐ₃ is selected from a group consisting of E, K, A, and conservative substitutions thereof; Xₐₐ₄ is selected from a group consisting of K, H, and conservative substitutions thereof; J_{N} comprises a component selected from a group consisting of hydrogen; poly(ethylene glycol); methyl; A, SA, LSA, E, AE, KAE, and conservative substitutions thereof; and combinations thereof; and, J_{C} comprises a component selected from a group consisting of hydroxyl; poly(ethylene glycol); amino; and G, GT, GTV, V, VT, VTV, and conservative substitutions thereof; and combinations thereof.

In some embodiments, the peptide comprises VARVELK (SEQ ID NO:7) or a conservatively modified variant thereof. In some embodiments, the peptide comprises VADEKLH (SEQ ID NO:8) or a conservatively modified variant thereof. In some embodiments, the peptide is further conjugated to a carrier peptide.

### Peptides Developed to Control δ-PKC/Annexin V Interactions

It has been recently discovered that δ-PKC is involved with the creation of tissue damage during ischemia and/or reperfusion. More specifically, inhibition of δ-PKC using a peptide inhibitor during simulated ischemia/reperfusion in isolated rat hearts has been shown to be cardioprotective (Inagaki, K. et al., Circulation, 108(19):2304 (2003); Inagaki, K. et al., Circulation, 108(7):869 (2003)). The peptides taught herein have been identified through a homology search between annexin V (SEQ ID NO:17) and δ-PKC (SEQ ID NO:16).

In some embodiments, the teaching includes a peptide comprising the amino acid sequence J_{N}-QXₐₐ₁EXₐₐ₂Xₐₐ₃A-J_{C} (SEQ ID NO:9), or a conservatively modified variant thereof, wherein Xₐₐ₁ is selected from a group consisting of V, A, and conservative substitutions thereof; Xₐₐ₂ is selected from a group consisting of Q, D, A, and conservative substitutions thereof; Xₐₐ₃ is selected from a group consisting of D, E, and conservative substitutions thereof; J_{N} comprises a component selected from a group consisting of hydrogen; poly(ethylene glycol); methyl; A, EA, DEA, L, SL, GSL, and conservative substitutions thereof; and combinations thereof; and, J_{C} comprises a component selected from a group consisting of hydroxyl; poly(ethylene glycol); amino; and Q, QA, QAL, N, NQ, NQP, and conservative substitutions thereof; and combinations thereof.

In some embodiments, the peptide comprises QVEQDA (SEQ ID NO:10) or a conservatively modified variant thereof. In some embodiments, the peptide comprises QAEDEA (SEQ ID NO:11) or a conservatively modified variant thereof. In some embodiments, the peptide is further conjugated to a carrier peptide.

Mutation of a charged residue in these peptides may be an important factor in the interaction between the annexin and the PKC proteins. The control peptides taught herein have a high homology to the inhibitor and activator peptides, but also have a mutation at the key residue and are expected to be the best control for measuring the efficacy of the activator and inhibitor peptides.

In some embodiments, the peptides have an optimal length of from about 5 to about 30 amino acids, from about 5 to about 20 amino acids, from about 5 to about 15 amino acids, from about 6 to about 20 amino acids, from about 6 to about 15 amino acids, from about 6 to about 10 amino acids, from about 6 to about 8 amino acids, or any range therein.

There are a number of assays that can be used to test the interaction between the annexin peptides taught herein and their respective PKCs. In some embodiments, the assays can determine a cellular response that includes a MARCKS phosphorylation, histone phosphorylation, and insulin-induced cell maturation. Other examples of assays are provided in the Examples section below.

A variety of cell types can be used in the assays and include established cell lines and primary cell cultures. Examples of cells that can be used to test the peptides taught herein include, but are not limited to, CHO cells which are hamster ovary cells useful, for example, in immunoprecipitation and translocation studies; PC3 cells which are prostate tumor cells useful, for example, in testing α-PKC activity; MCF7 cells which are human breast cancer cells useful, for example, in testing α-PKC activity; adult neonatal cardiomyocytes useful, for example, in testing cardiac hypertrophy and β-PKC activity; HeLa cells which are human cervical cancer cells useful for testing α-PKC activity, translocation and immunoprecipitation; and xenopus oocytes which is useful for testing β-PKC activity.

The skilled person will appreciate that various changes can often be made to the amino acid sequence of a polypeptide which has a particular activity to produce variants (sometimes known as derivatives or “muteins”) having at least a proportion of the desired biological activity, and preferably having a substantial proportion of said activity. The skilled person is aware that various amino acids have similar properties. One or more such amino acids of a substance can often be substituted by one or more other such amino acids without eliminating a desired activity of that substance.

Amino acid substitutions can be made in the peptides taught herein. In some embodiments, the amino acids glycine, alanine, valine, leucine and isoleucine can be substituted for one another (amino acids having aliphatic side chains). Of these possible substitutions, it is sometimes preferred that glycine and alanine substitute for one another (since they have relatively short side chains) and that valine, leucine and isoleucine substitute for one another (since they have larger aliphatic side chains which are hydrophobic). In some embodiments, other amino acids that can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur-containing side chains).

Amino acid deletions or insertions may also be made relative to a desired amino acid sequence. For example, amino acids which do not have a substantial effect on the activity of the peptide, or at least which do not eliminate such activity, may be deleted. Such deletions can be advantageous since the overall length and the molecular weight of a peptide can be reduced whilst still retaining activity. This can enable the amount of peptide required for a particular purpose to be reduced and, potentially reduce the required dosage levels.

Whatever amino acid changes are made (whether by means of substitution, insertion, or deletion), the peptides taught herein have at least 50%, at least 75%, at least 80%, at least 85%, at least 90%, at least 95%, at least 98%, or any range percentage therein, of a sequence identity with the desired peptide.

The annexin peptides, and their variants and conservatively modified variants, may be produced using a variety of methods known to those skilled in the art. In some embodiments, chemical methods can be used to synthesize the amino acid sequences. For example, peptide synthesis can be performed using various solid-phase techniques (Roberge, J. Y. et al., Science, 269:202 (1995)) and automated synthesis may be achieved, for example, using a synthesizer, such as the ABI 431A Peptide Synthesizer (Perkin Elmer). The synthesized peptide may be substantially purified by preparative high performance liquid chromatography (e.g., Creighton, T. (1983) Proteins, Structures and Molecular Principles, WH Freeman and Co., New York, N.Y.). The composition of the synthetic peptides may be confirmed by amino acid analysis or sequencing (e.g., the Edman degradation procedure; Creighton, supra). Additionally, the amino acid sequence of the annexin peptide, or any part thereof, may be altered during direct synthesis and/or combined using chemical methods with sequences from other proteins, or any part thereof, to produce a variant polypeptide.

The teaching also contemplates a modified annexin peptide, where the annexin peptide, and any of the variants or conservatively modified variants thereof, can be extended on the C-terminus or N-terminus by a chemical moiety. Examples of such post-translational modifications in the teachings can include, but are not limited to, the addition of modifying groups described below through processes such as, for example, glycosylation, acetylation, phosphorylation, modifications with fatty acids, formation of disulfide bonds between peptides, biotinylation, PEGylation, methylation, biotinylation, amination, adding a biochemical label, radiolabel, and the like, all of which are techniques well known to those skilled in the art. The peptide can be modified, for example, with a polyalkylene glycol, such as poly(ethylene glycol) (PEG) using a variety of techniques known to one of skill in the art. There are a variety of available PEG sizes and derivatives that are commercially designed for specific applications such as, for example, attachment to a variety of different chemical functionalities including, but not limited to, amines, thiols, hydroxyls, sulfhydryls, and carboxyls.

In some embodiments, a modified annexin for use as described herein can be extended on the N-terminus with an amino acid residue that provides an accessible sulfhydryl group. Such a modified annexin can be conjugated to any of a number of carrier peptides to assist in transport of the annexin peptide across a cell membrane.

Any appropriate carrier peptide known to one of skill in the art can be used in the teachings provided. Examples of such carrier peptide can include, but are not limited to, TAT (SEQ ID NO:4), Drosophila Antennapedia homeodomain (SEQ ID NO:5), or other oligo- or polypeptides that can be homo- or hetero- in amino acid content, such as a polyarginine homopolymer or oligomer. Methods of attaching these carriers are well-established in the art, for example, the annexin peptide can include one or more cysteine residues added to the N-terminus or to the C-terminus. The sulfhydryl group or groups of the added cysteine residue or residues can be joined to a carrier peptide, such as TAT (SEQ ID NO:4), Drosophila Antennapedia homeodomain (SEQ ID NO:5); Théodore, L. et al. J. Neurosci., 15:7158 (1995); Johnson, J. A. et al., Circ. Res. 79:1086 (1996)) or to a polyarginine (Mitchell et al., J. Peptide Res., 56:318-325 (2000); Rothbard et al., Nature Med., 6:1253-1257 (2000)).

Those skilled in the art will appreciate that the teachings also encompass polynucleotides which encode the peptides taught herein, as well as their variants and conservatively modified variants. Accordingly, any nucleic acid sequence which encodes the amino acid sequences described herein can be used to produce recombinant molecules which express the peptides. It will be appreciated by those skilled in the art that as a result of the degeneracy of the genetic code, a multitude of nucleotide sequences can be used for a single amino acid sequence, some bearing minimal homology to the nucleotide sequences of any known and naturally occurring gene. Thus, the teaching contemplates each and every possible variation of nucleotide sequence that could be made by selecting combinations based on possible codon choices. These combinations are made in accordance with the standard triplet genetic code, and all such variations are to be considered as being specifically disclosed.

### III. Compositions and Methods of Treatment

The teaching also contemplates a method of regulating the PKC-mediated cellular responses by administering an annexin peptide having a sequence corresponding to the sequences described herein, or a sequence having greater than or at least about 50% identity to those sequences, more preferably 75% identity, still more preferably 80%, 85%, 90%, or 95% identity to those sequences. In the method, the peptide is administered in a suitable formulation, where the peptide is effective to bind to a PKC and modulate an interaction between an annexin and the PKC in vivo.

A pharmaceutical composition comprising an annexin peptide, or a variant thereof, is also contemplated. The pharmaceutical composition comprises the peptide in combination with a carrier, selected in part according to the desired route of administration. Any number of routes of administration are contemplated, including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Suitable pharmaceutically-acceptable carriers comprise excipients and auxiliaries which facilitate processing of the active compounds into preparations. Further details on techniques for formulation and administration may be found in the latest edition of Remington's Pharmaceutical Sciences (Maack Publishing Co., Easton, Pa.). For example, pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions taught herein may be manufactured in a manner that is known in the art, e.g., by means of conventional mixing, dissolving, granulating, dragee-making, levigating, emulsifying, encapsulating, entrapping, or lyophilizing processes. After pharmaceutical compositions have been prepared, they can be placed in an appropriate container and labeled for treatment of an indicated condition. For administration of an annexin peptide, such labeling would include amount, frequency, and method of administration.

Pharmaceutical compositions suitable for use in the teachings include compositions wherein the active ingredients are contained in an effective amount to achieve the intended purpose. The determination of an effective dose is well within the capability of those skilled in the art. For example, the therapeutically effective dose can be estimated initially either in cell culture assays or in animal models, usually mice, rabbits, dogs, or pigs. The animal model may also be used to determine the appropriate concentration range and route of administration. Such information can then be used to determine useful doses and routes for administration in any mammal including, but not limited to, humans.

A therapeutically effective dose refers to that amount of active ingredient, such as an annexin peptide or variant thereof, which prevents, inhibits, or ameliorates the symptoms, or the condition itself, in a patient. The data obtained from cell culture assays and animal studies is used in formulating a range of dosage for therapeutic use in a subject, where the term “subject” and “patient” can be used interchangeably in these teachings and refer to an animal such as a mammal including, but not limited to, non-primates such as, for example, a cow, pig, horse, cat, dog, rat and mouse; and primates such as, for example, a monkey or a human. The dosage contained in such compositions will often be within a range of circulating concentrations that provides therapeutic efficacy with little or no toxicity. The dosage can vary within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. In some embodiments, exemplary therapeutically effective amounts of a peptide (i.e., an effective dosage) ranges from about 0.001 to 30 mg/kg body weight, preferably about 0.01 to 25 mg/kg body weight, more preferably about 0. 1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. Treatment of a subject with a therapeutically effective amount of the peptide can include a single treatment or, preferably, can include a series of treatments.

The teaching contemplates several therapeutic uses, some of which are taught herein, and others which will be apparent to one of skill. In some embodiments, the teaching includes a method of increasing or decreasing a cellular response to β-protein kinase C (β-PKC) (βI is SEQ ID NO:12; βII is SEQ ID NO:34), wherein the method comprises adding a peptide from annexin I (SEQ ID NO:13) to an environment comprising a cell, annexin I, and β-PKC; wherein, the peptide binds to the β-PKC and inhibits binding between the β-PKC and the annexin I. In some embodiments, the cellular response includes translocation of the β-PKC from the cytosolic fraction of cell to the particulate fraction of the cell. In some embodiments, the cellular response includes cellular hypertrophy. In some embodiments, the cellular response includes hyperproliferation.

In some embodiments, the teaching includes a method of increasing or decreasing a cellular response to α-protein kinase C (α-PKC) (SEQ ID NO:14), wherein the method comprises adding a peptide from annexin VI (SEQ ID NO:15) to an environment comprising a cell, annexin VI, and α-PKC; wherein, the peptide binds to the α-PKC and inhibits binding between the α-PKC and the annexin VI. In some embodiments, the cellular response includes translocation of the α-PKC from the cytosolic fraction of cell to the particulate fraction of the cell. In some embodiments, the cellular response includes hyperproliferation.

In some embodiments, the teaching includes a method of increasing or decreasing a cellular response to δ-protein kinase C (δ-PKC) (SEQ ID NO:16), wherein the method comprises adding a peptide from annexin V (SEQ ID NO:17) to an environment comprising a cell, annexin V, and δ-PKC; wherein, the peptide binds to the δ-PKC and inhibits binding between the δ-PKC and the annexin V. In some embodiments, the cellular response includes translocation of the δ-PKC from the cytosolic fraction of cell to the particulate fraction of the cell. In some embodiments, the cellular response includes cardioprotection following an ischemia/reperfusion injury. In these embodiments, the methods can be directed to protecting tissue against damage due to the ischemia/reperfusion injury. In this method, a patient at risk of suffering ischemic damage can be treated with an annexin V peptide, or a variant or a modification thereof, to induce protection against ischemic damage.

### IV. Examples

The following examples further illustrate the teachings described herein and are in no way intended to limit the scope of the claims.

### Example 1

### Binding of δ-PKC to Annexin V

This example demonstrates that a PKC and an annexin component within a given PKC/annexin pair will bind with each other and, more specifically, shows that δ-PKC (SEQ ID NO:16) binds to annexin V (SEQ ID NO:17). A binding assay was performed in vitro, using an immobilized annexin V with the δ-PKC, δ-V1 (SEQ ID NO:18) (the C2 domain of δ-PKC), δ-V5 (SEQ ID NO:19), and ε-V1 (SEQ ID NO:20) peptides. These domains are recognized in the art as participating in protein-protein interactions.

10 μg of the annexin V was separated on 12% SDS-PAGE gel and transferred to nitrocellulose, and then cut into strips. The strips were incubated with blocking buffer (50 mM Tris pH 7.5, 200 mM NaCl, 3% bovine serum albumin, 0.1% PEG20). The strips were then incubated in overlay buffer (50 mM Tris pH 7.5, 200 mM NaCl, 1% PEG20, 12 mM β-mercaptoethanol) in the presence where appropriate of 30 μg crude bacterial cell lysate containing fusion proteins of myelin basic protein (MBP)-delta V1 PKC (MBP-δV1), glutathione-S-transferase-delta V5 PKC (GST-δV5), and full length sequences of δ-PKC or rat purified ε-PKC (SEQ ID NO:21) at 37° C. for 30 min. The strips where then washed with wash buffer (50 mM Tris pH 7.5, 200 mM NaCl, 0.1% PEG20, 12 mM β-mercaptoethanol), and then incubated with fixative (0.2% formaldehyde in PBS) and neutralizing solution (0.2M glycine in PBS) at room temperature 20 min each. After washing with buffer, the strips were probed with antibodies according to the overlay protein, and resulting bands were visualized using ECL Western blotting.

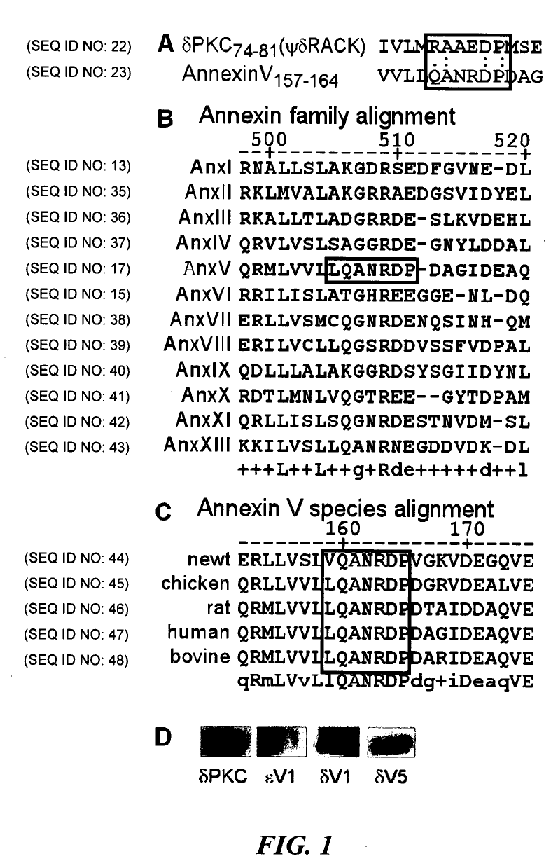

FIGS. 1 A-D show that the domains V1/C2 and V5 of δ-PKC, as well as full-length δ-PKC, were observed to bind to the annexin V. FIG. 1A shows a sequence similarity between δ-PKC residues 74-81, MRAAEDPM (SEQ ID NO:22) (“ψδRACK”) and annexin V residues 157-164, LQANRDPD (SEQ ID NO:23). FIG. 1B shows that no other member of the annexin family has this sequence similarity. FIG. 1C shows that this annexin V sequence is conserved between species.

The results in FIG. 1D indicate that the full-length δ-PKC, as well as its V1 and V5 domains, bind to the annexin V to form a complex. In contrast, FIG. 1D also shows that the V1 region of another member of the novel PKC isozymes, the ε-PKC (SEQ ID NO:21), did not bind to the annexin V, indicating that the selectivity in this interaction.

### Example 2

### Timing of the Binding of δ-PKC to Annexin V in a Cell

This example shows that, in the absence of over-expression of any of the proteins in questions, the δ-PKC (SEQ ID NO:16) and annexin V (SEQ ID NO:17) bind to form a complex in a cell. The complexes were immunoprecipitated with anti-δ-PKC antibodies and probed for the presence of annexin V at the combined molecular weight of about 110 kD.

CHO-K1 cells (available through the ATCC) were grown in F-12 (HAM) nutrient mixture supplemented with GLUTAMAX, 10% fetal bovine serum, and antibiotics (100 units/ml penicillin and 100 mg/ml streptomycin sulfate, all from Invitrogen Corp.). The cells were cultured at 37° C. under 5% CO2. At 48 hours prior to the study, the cells were serum starved. They were stimulated with 10 nM phorbol 12-myristate 13-acetate (PMA) (available through LC Laboratories) or with 5 mM H₂O₂ (available through Sigma Chemical Corp.) for the indicated duration ranging from about 30 seconds to about 5 minutes, a technique well-known to one of skill in the art. The cells were washed with cold PBS, and homogenized on ice with trituration in homogenization buffer (20 mM Tris pH 7.4, 2 mM EDTA, 10 mM EGTA, 0.25M sucrose, 12 mM β-mercaptoethanol, protease inhibitor cocktail (available through Sigma Chemical Corp)), 0.1% TRITON X-100, and 1% formaldehyde.

It was observed that the 0.1% TRITON extracts all of the translocatable PKC from the cell. After 30 min at 4° C., the lysates were quenched with 0.14M glycine for 20 min at 4° C., and the samples were spun at 14K rpm at 4° C. The supernatant was incubated with 1 μg anti-δ-PKC antibody (available through Santa Cruz Biotechnology) for 1 hour, followed by exposure to protein-G beads (available through Invitrogen Corp.) for 3 hours at 4° C. The beads were then washed with wash buffer (20 mM Tris pH 7.5, 2 mM EDTA, 100 mM NaCl, 12 mM β-mercaptoethanol, 0.1% TRITON), and separated on 7.5% SDS-PAGE, transferred to nitrocellulose, and probed with anti-annexin V (available through Santa Cruz Biotechnology), followed by visualization using an enhanced chemiluminescence (ECL) method.

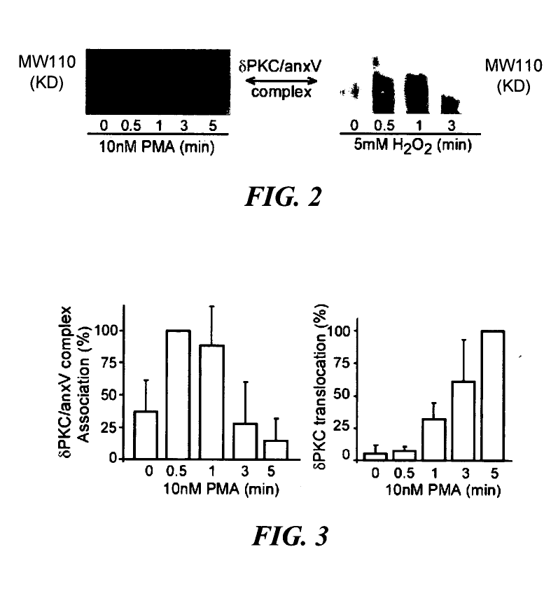

FIG. 2 shows the effect of stimulation on the formation of a complex between δ-PKC and annexin V. The molecular weight of the complex is about 110 kDaltons (kD), since the δ-PKC is about 73 kD and the annexin V is about 35 kD molecular weight. As shown in FIG. 2, stimulation of the CHO cells with 10 nm PMA caused about a 4-fold increase in the amount of annexin V/δ-PKC complex that formed within the first 0.5 minutes of treatment. The interaction was short-lived, and after 1 minute of stimulation the amount of annexin V/δ-PKC complex was reduced to levels below that which are obtained prior to PMA treatment. A transient association between the annexin V and δ-PKC was also observed following activation of δ-PKC with H₂O₂ (5 mM, 0.5-1 minute treatment). Therefore, it was apparent the annexin V and δ-PKC began complexing early after cell stimulation, regardless of the means used to stimulate the δ-PKC.

### Example 3

### The Timing of the Binding of δ-PKC to Annexin V Relative to Translocation of the δ-PKC in a Cell

Activation of PKCs results in their translocation within a cell. The timing of the formation of a PKC/annexin complex, particularly a complex of δ-PKC and annexin V (SEQ ID NO:17), was compared relative to the timing of the translocation of the δ-PKC from the cytosolic fraction of the cell to the particulate fraction of the cell.

The CHO cells of Example 1 were fractionated, stimulated, and washed with cold PBS, scraped in homogenization buffer, and spun at 100,000 g for 30 min at 4° C., resulting in the soluble fraction. The pellet was then resuspended in a homogenization buffer with 1% TRITON X-100, and spun under the same conditions. Where applicable, the cells were pre-incubated with 1 μM peptide for 15 minutes prior to stimulation. The samples were then analyzed using a Western blot, and the loading was corrected for protein concentration using an internal control such as actin.

FIG. 3 shows the timing of the binding of δ-PKC to annexin V relative to translocation of the δ-PKC in a cell. While a maximum annexin V/δ-PKC complex accumulation was found between about 0.5 minutes and about 1 minute, translocation of δ-PKC from the soluble cytosolic fraction of the cell to the particulate fraction of the cell occurred later; it began only after about 1 minute of PMA stimulation and reached a maximum after about 5 minutes of stimulation. This shows that formation of the δ-PKC/annexin V complex precedes δ-PKC translocation from the cytosolic fraction of the cell to the particulate fraction of the cell.

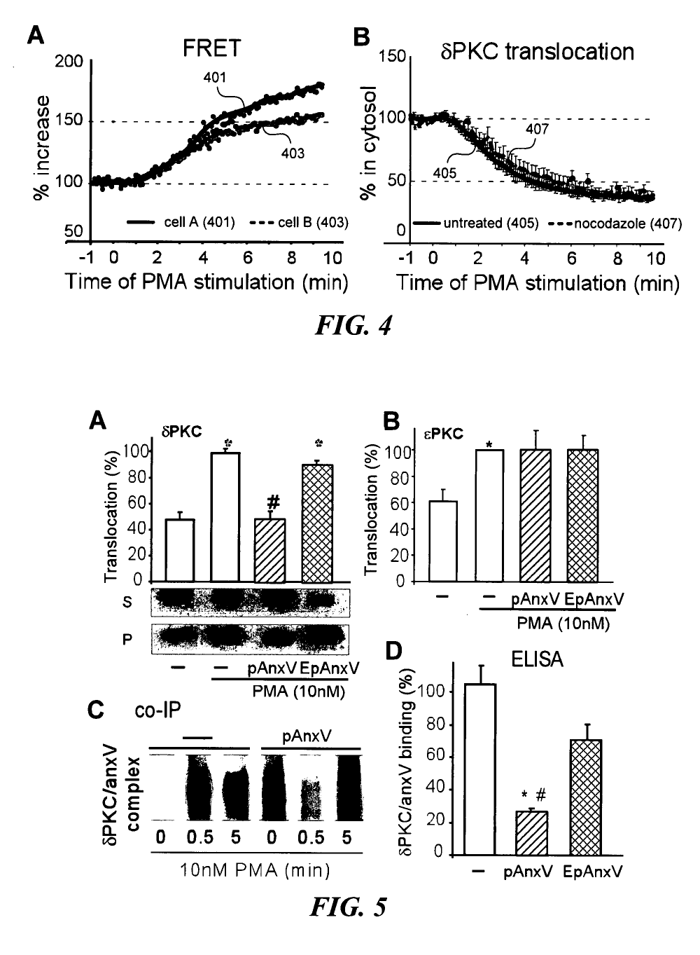

FIGS. 4A and 4B show the results of a real-time fluorescent imaging of the interaction of δ-PKC and annexin V in a cell. Fluorescence Resonance Energy Transfer (FRET) was used to verify the immunoprecipitation results shown in FIG. 3 by stimulating the CHO cells with PMA and measuring the FRET within the cells. Pixel intensity was monitored in the selected regions as a function of time after 100 nM PMA stimulation. The FRET signal indicates the interaction of δ-PKC and annexin V and is seen to increase upon PMA stimulation in a time dependent manner.

CHO cells were grown on chambered #1 Borosilicate coverglass (Lab-Tek), transfected at 50% confluency using Fugene 6 (Roche), according to the manufacturer's instructions, with CFP-annexin V and YFP-δPKC cloned into the pECFP-C1 and pEYFP-C1 vectors, respectively (Clontech). The cells were serum starved for 24 hours. Real-time confocal imaging was conducted on a spinning disk Nipkow confocal microscope, and the cells were viewed using an inverted Olympus IX70 microscope with a 40× oil immersion Olympus objective (1.35 NA). Images were acquired using a CCD camera (Hamamatsu) with 2×2 pixel binning. The CFP was excited with the 442 nm laser line of a helium-cadmium laser (Kimmon), whereas the YFP was imaged with the 514 nm line of an argon ion laser (Melles-Griot). The images were acquired at 27° C. every 5-10 sec for 10 minutes, and 100 nM PMA was added to the cell chamber after the 10th image in each time series. Where indicated, the cells were pretreated with nocodazole (10 μM, 30 min). Exposure time was adjusted for photobleaching levels lower than 10%.

Fluorescence intensity was measured using METAMORPH data analysis software (Universal Imaging). To monitor the translocation of the PKC and the increases in FRET signal, a small region of interest was selected from each cell and fluorescence intensity values were graphed against time and normalized to the initial fluorescence. FRET was calculated using a formula that corrects for bleed-through and donor concentration: FRET=(I(₄₄₂₋₅₃₀)-αI(₄₄₂₋₄₈₀)-βI(₅₁₅₋₅₃₀))/I(₄₄₂₋₄₈₀). The α and β coefficients were calculated from singly transfected cells, where α=I(₄₄₂₋₅₃₀)/I(₄₄₂₋₄₈₀)=47.6% of CFP bleed-through into the YFP channel, and β=I(₄₄₂₋₅₃₀)/I(₅₁₅₋₅₃₀)=14.4% of YFP excitation from the CFP channel.

Whereas annexin V remains cytosolic throughout the experiment, δ-PKC translocation from the cytosol to the membrane can be observed starting from 2 minutes of stimulation and reaches its maximal translocation by 9 min, as shown in FIG. 4B, which represents results from an average of 6 cells from 3 independent experiments. Note that a 10 μM nocodazole pretreatment (407) for 30 minutes did not alter the translocation profile of overexpressed δ-PKC when compared to untreated conditions (405). As shown in FIG. 4A, cell A (401) and cell B (403) are regions that were chosen for quantifying the FRET signal during imaging, and the results are representative of 30 different cells from 10 independent experiments. The FRET signal increased predominantly in the cytosolic region of the stimulated cells, and the basal FRET level at the cell membrane was higher than found in the cytosol but did not increase after cell stimulation. Interestingly, the time-course of FRET increase followed the timecourse of δ-PKC translocation, as shown by comparing FIGS. 4A and 4B. Importantly, the fold increase of the FRET signal shown in FIG. 4A was comparable to the fold increase of complex association as seen in the co-immunoprecipitation study shown in FIG. 3.

### Example 4

### A Peptide Designed to Block δ-PKC/Annexin V Complex Formation Blocks δ-PKC Translocation

Example 3 shows that δ-PKC/annexin V binding occurs prior to accumulation of δ-PKC at the particulate fraction. One of skill will appreciate that peptides derived from PKC-binding proteins can inhibit PKC translocation and function. This example shows that an interaction between the annexin V and δ-PKC is required for δ-PKC translocation through the use of a peptide that was designed to specifically block this interaction.

If annexin V interaction with δ-PKC is required for δ-PKC translocation, the annexin V-derived peptide should block a stimulation-induced translocation of δ-PKC by binding to the δ-PKC and preventing its association with annexin V. In this example, a peptide corresponding to the δ-PKC homology site on annexin V is used to inhibit δ-PKC translocation and function. The length of the annexin-derived peptide (“the annexin-derived peptide”, “AinxV₁₅₇₋₁₆₄”, or “pAnxV”) (SEQ ID NO:22) has been extended by one amino acid at the N-terminal region and C-terminal region to correspond to the length of the ψδRACK peptide (SEQ ID NO:23) shown in FIG. 1A. This modification is also designed to prevent any potential effects of the free carboxyl and amino groups at the ends of the peptide. The annexin V-derived peptide was synthesized and conjugated to a TAT₄₇₋₅₇ carrier peptide (SEQ ID NO:4) to enable intracellular delivery of the peptide across biological membranes.

FIGS. 5A-D show the effect of a peptide designed to inhibit the translocation of δ-PKC. As shown in FIG. 5A, pretreatment of CHO cells with 1 μM pAnxV blocked PMA-induced translocation of δ-PKC to the particulate fraction after 5 minutes of PMA stimulation. There is a charge difference in the homologous sequence between annexin V and δ-PKC(R₁₆₁ to E₇₈, respectively), similar to what has been seen in all the homologous sequences between PKCs and their binding proteins. As expected and, as shown in FIG. 5B, when the arginine in annexin V was replaced with the glutamate found in δ-PKC to create the EpAnxV peptide (SEQ ID NO:33), the inhibitory effect on δ-PKC translocation was lost. Importantly, neither peptide affected PMA-induced translocation of ε-PKC (SEQ ID NO:21), indicating the selectivity of the annexin V-derived peptide effect on δ-PKC translocation.

The pAnxV peptide was also tested in the immunoprecipitation studies as described in Example 2. As shown in FIG. 5C, the pAnxV peptide blocked the δ-PKC/annexin V complex formation. Furthermore, and as shown in FIG. 5D, the pAnxV inhibited δ-PKC/annexin V binding in an ELISA assay, whereas the EpAnxV had no significant effect. Together, these data suggest that the pAnxV peptide corresponds to at least a part of the δ-PKC-binding site on annexin V, and that an annexin V/δ-PKC interaction is required for stimulation-induced δ-PKC translocation from the cytosolic fraction to the cell particulate fraction in a cell.

Note that in FIGS. 5A and 5B, the * is at p<0.001 for—vs. PMA alone (“-” is no peptide treatment), and the # is at p<0.001 for PMA vs. pAnxV+PMA. In FIG. 5D, the * is at p<0.006 for pAnxV vs.—(“-” is no peptide treatment), and the # is at p<0.007 for pAnxV vs. EpAnxV. The representative blot of δ-PKC translocation from soluble (S) to the particulate (P) fraction is shown.

### Example 5

### δ-PKC/Annexin V Complex Formation is Required for δ-PKC Function In Vivo

The next question includes whether δ-PKC/annexin V complex formation described above is required for δ-PKC-mediated function, in vivo. One of skill should appreciate that inhibition of δ-PKC translocation can inhibit cardiac damage. Since our data suggest that interaction of δ-PKC with annexin prior to translocation is essential in the process of translocation, the annexin V-derived peptide could also be cardioprotective when administered prior to δ-PKC activation.

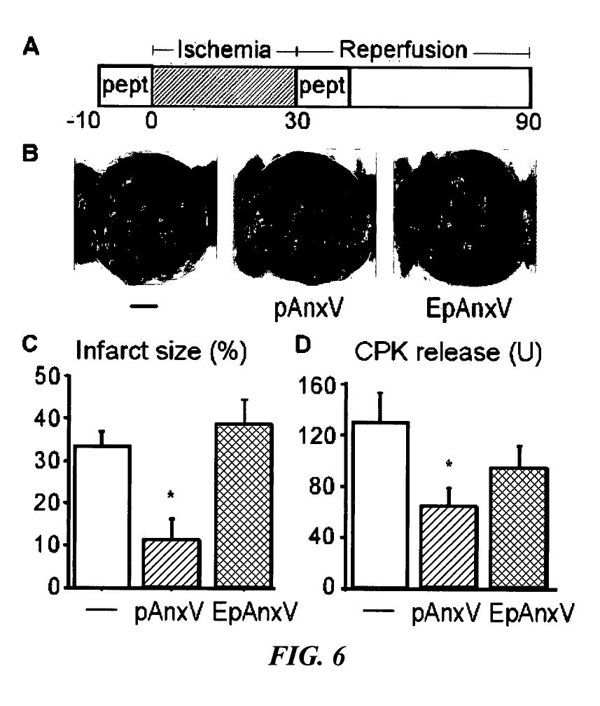

FIGS. 6 A-D show that association of δ-PKC with annexin V is needed for downstream signaling in an ex vivo model of ischemia and reperfusion. FIG. 6A shows that isolated rat hearts were perfused with pAnxV or EpAnxV (1 μM) for 10 minutes prior to a 30-minute no-flow ischemia and for 10 minutes at the onset of 60 minutes of reperfusion. FIG. 6B shows that the hearts were sliced and stained with 2,3,5-triphenyl-2H-tetrazolium chloride (TTC) to visualize live tissue and dead tissue. FIG. 6C compares the infarct sizes that were measured on both sides of the tissue slices and averaged. The pAnxV treatment reduced infarct size by about 70%, whereas the EpAnxV had no effect. In FIG. 6D, creatine phosphokinase (CPK) release was measured in the heart perfusate during the reperfusion. Treatment with pAnxV resulted in a greater than 50% reduction in necrosis, whereas treatment with the EpAnxV did not show any statistical difference from the control heart.

The hearts treated with the annexin V-derived peptide, pAnxV, prior to ischemia, had a 70% reduction in infarct size and ˜50% reduction in cell necrosis as measured by the leakage of the cardiac enzyme, creatine phosphokinase (CPK), into the heart perfusate. Note that EpAnxV, which did not inhibit δ-PKC translocation as described above, also did not inhibit ischemia-induced δ-PKC-mediated damage to the heart. These data indicate that an interaction between δ-PKC and annexin V is part of δ-PKC function, and confirms the importance of the formation of a complex between δ-PKC and annexin V as an initial step for δ-PKC function in vivo.

### Example 6

### Other δ-PKC/Annexin V Sequence Homologies can Affect Translocation and Provide Cardioprotection

Examples 1-5 provide a test model for the use of an annexin peptide designed from a sequence homology between a PKC and the annexin to control the translocation of the PKC and, thus, the activity of the PKC in a cell. This example describes the testing of peptides identified by evaluating homology between δ-PKC (SEQ ID NO:16) and annexin V (SEQ ID NO:17):

| | |
|---|---|
| annexin V residues 168-180: | DEAQVEQDAQALF |
| (inhibitor) | (SEQ ID NO: 24) |
| δ-PKC residues 15-27: | GSLQAEDEANQPF |
| (activator) | (SEQ ID NO: 25) |

The peptides will be designed from the following subsets of the homology:

| | |
|---|---|
| pAnxV | QVEQDA |
| | (SEQ ID NO: 10) |
| ΨAnxV (from δ-PKC) | QAEDEA |
| | (SEQ ID NO: 11) |
| cAnxV (control peptide) | QVEADA |
| | (SEQ ID NO: 26) |

The assays described in the above examples can be applied to confirm the effects on translocation and cardioprotection, for example, in treatments directed to ischemia and reperfusion.

### Example 7

### δ-PKC/Annexin I Sequence Homologies can Affect Translocation, Control Cardiac Hypertrophy, and Serve as a Therapeutic in Cancer Treatments

This example describes the testing of peptides developed using a newly discovered homology between the β-PKC (βI is SEQ ID NO:12; βII is SEQ ID NO:34) and the annexin I (SEQ ID NO:13):

| | |
|---|---|
| annexin I residues 131-142: | LGTDEDTLIEIL |
| (inhibitor) | (SEQ ID NO: 27) |
| β-PKC residues 166-177: | AHIDRDVLIVLV |
| (activator) | (SEQ ID NO: 28) |

Peptides are designed from the following subsets of the homology:

| | |
|---|---|
| pAnxI | DEDTLI |
| | (SEQ ID NO: 2) |
| ΨAnxI (from β-PKC) | DRDVLI |
| | (SEQ ID NO: 3) |
| cAnxI (control peptide) | DADVLI |
| | (SEQ ID NO: 29) |

The assays described above are applied to confirm the effects of the annexin I peptides on translocation and cardiac hypertrophy. The assays can include (i) binding assays to assess the binding of β-PKC to annexin I, including competitive binding with the select annexin I peptides; (ii) translocation assays in the presence of PMA and the select annexin I peptides; (iii) substrate phosphorylation assays using a MARCKS substrate; (iv) assessment of the effects of the select peptides on insulin-induced cell maturation; (v) assessment of the effects of the select peptides on cardiac hypertrophy in cardiac myocytes; (vi) assessment of the effects of the select peptides on tumor growth and angiogenesis; and (vii) an assessment of the effects of the select peptides on the inhibition of the transition to heart failure.

The binding of β-PKC to annexin I can be tested using in vitro binding assays of β-PKC (both I and II) to annexin I. As described above, peptides designed to block δ-PKC/annexin V interaction block the binding of δ-PKC to annexin V as seen both by co-immunoprecipitation studies and ELISA assay. The testing can be conducted in the presence and absence of the select annexin I peptides. The binding of β-PKC I and II to annexin I will be tested in the presence and absence of the peptides in ELISA, pull-down studies, and co-immunoprecipitations from cells. The pAnxI is expected to block the binding of bPKC to annexin I, while ψAnxI is expected to promote the binding of bPKC to annexin I. The control peptide should have no activity. The requirement for PKC activators, such as calcium, DAG, and PS, will be assessed in these assays to determine whether the binding of β-PKC and annexin I occurs when the PKC is active, inactive, or in the process of activation.

The translocation of β-PKC can be tested using PMA induced translocation of the PKC from the soluble, or cytosolic, fraction to the membrane, or particulate, fraction. The translocation of beta I and beta II PKC can be assessed in the presence of PMA and the above-described peptides. The pAnxI is expected to block δ-PKC translocation, while ψAnxI peptide is expected to promote β-PKC translocation. The control peptide cAnxI should have no affect on β-PKC translocation.

The substrate phosphorylation can be determined using MARCKS as the substrate, since it is a known PKC substrate in cells. In order to assay the peptides described above, cells from wildtype and β-PKC knockout animals can be be tested for MARCKS phosphorylation in the presence of the select peptides. We expect that, if the peptides are specific for β-PKC, there will be no peptide effect in β-PKC knockout cells, while pAnxI is expected to inhibit MARCKS phosphorylation in wildtype cells. In contrast, ψAnxI peptide will promote MARCKS phosphorylation in wildtype cells, but not in knockout cells. The cAnxI is not expected to affect the outcome of the assay.

The effect of the select peptides in cardiac hypertrophy can be tested using neonatal cardiac myocytes treated with phenylephrine. The myocytes will develop the phenotype of hypertrophy, or increase in cell size. The β-PKC has been shown to regulate this process, and inhibition of the β-PKC function through the disruption of its interaction with RACK using rationally designed peptides has previously been shown to block the hypertrophy (Stebbins JBC 2001 Aug. 10; 276(32):29644-50). Cell size may be measured either directly by taking photographs of cells and measuring their footprint area, and it may also be indirectly measured through isotope labeled phenylalanine incorporation differences, or atrial natriuretic factor (ANF) staining as marker of hypertrophy, both of which are good measures of hypertrophy. It is expected that pAnxI will block hypertrophy, while ψAnxI will increase it. The cAnxI should have no effect on the outcome of the study.

The role of β-PKC in the maturation of xenopus oocytes can be measured, since inhibition of β-PKC has been shown to inhibit insulin-induced maturation of xenopus oocytes (Ron JBC 1994 Aug. 26; 269(34):21395-8). It is expected that pAnxI will inhibit both the translocation of bPKC, as well as oocyte maturation, while ψAnxI will promote both. The cAnxI is not expected to affect the outcome of the study.

The role of β-PKC in cancer and angiogenesis can be determined, since β-PKC has been shown to play a role in both tumor growth and angiogenesis. A human prostate cancer mouse model has shown that inhibition of β-II-PKC blocks tumor growth. The peptides described above will be administered in a continuous fashion through an implanted pump, and tumor size can be assayed at different points of time to determine progression. In addition, angiogenesis can be measured at the same time points. It is expected that pAnxI will slow the tumor growth, and decrease angiogenesis, while ψAnxI will increase tumor growth rate and increase angiogenesis. The cAnxI peptide should not affect the outcome of the study.

The ability of the peptides to inhibit a transition to heart failure can be determined. Dahl salt rats transition from hypertrophy to heart failure, and this process has been shown to be dependent on β-II-PKC. The peptides described above will be administered in a continuous fashion through an implanted pump. The endpoint life duration, as well as the heart function throughout the experiment, can be measured. It has been observed that the inhibition of β-II-PKC prolongs the life and improves cardiac function in the Dahl salt model. It is expected that the pAnxI peptide will extend the life and improve cardiac function throughout. The ψAnxI peptide is expected to shorten the lifespan and decrease heart function. The cAnxI peptide should have no affect on the outcome of the study.

### Example 8

### α-PKC/Annexin VI Sequence Homologies can Affect Translocation and Serve as a Therapeutic in Cancer Teatments

This example describes the testing of peptides developed using a newly discovered α-PKC/annexin VI homology between the α-PKC (SEQ ID NO:14) and the annexin VI (SEQ ID NO:15):

| | |
|---|---|
| annexin VI residues 345-365: | LSAVARVELKGTVRPANDFNP |
| (inhibitor) | (SEQ ID NO: 30) |
| α-PKC residues 168-185: | KAEVADEKLHVTVRDAKNLIP |
| (activator) | (SEQ ID NO: 31) |

The annexin VI peptides will be designed from the following subsets of the homology:

| | |
|---|---|
| pAnxVI | VARVELK |
| | (SEQ ID NO: 7) |
| ΨAnxVI (from α-PKC) | VADEKLH |
| | (SEQ ID NO: 8) |
| cAnxVI (control peptide) | VAAVALK |
| | (SEQ ID NO: 32) |

The assays described above can be applied to confirm the effects of the annexin VI peptides on translocation and cancer. The assays can include (i) binding assays to assess the binding of α-PKC to annexin VI, including competitive binding with the select annexin VI peptides; (ii) translocation assays in the presence of PMA and the select annexin VI peptides; (iii) substrate phosphorylation assays using a MARCKS substrate; and (iv) assessment of the effects of the select peptides on cancer.

The binding of α-PKC to annexin VI can be tested using in vitro binding assays and will be conducted in the presence and absence of the annexin VI peptides. As described above, peptides designed to block δ-PKC/annexin V interaction block the binding of δ-PKC to annexin V as seen both by co-immunoprecipitation studies and ELISA assay. Therefore, the binding of α-PKC to annexin VI will be tested in the presence and absence of peptides in ELISA, pull-down studies, and co-immunoprecipitations from cells. It is expected that the pAnxVI will block the binding of α-PKC to annexin VI, while ψAnxVI will promote the binding of α-PKC to annexin VI. The control peptide should have no activity. The requirement for PKC activators, such as calcium, DAG, and PS, will be assessed in these assays to determine whether the binding of α-PKC and annexin VI occurs when the PKC is active, inactive, or in the process of activation.

The translocation of α-PKC can be tested using PMA induced translocation of the PKC from the soluble, or cytosolic, fraction to the membrane, or particulate, fraction. The translocation of α-PKC will be assessed in the presence of PMA and the above-described annexin VI peptides. It is expected that the pAnxVI will block α-PKC translocation, while ψAnxVI peptide will promote aPKC translocation. The control peptide cAnxVI should have no affect on α-PKC translocation.

The substrate phosphorylation can be determined using MARCKS as the substrate, since it is a known PKC substrate in cells. In order to assay the peptides described above, cells from wildtype and α-PKC knockout animals can be be tested for MARCKS phosphorylation in the presence of the select peptides. We expect that, if the peptides are specific for α-PKC, there will be no peptide effect in α-PKC knockout cells, while pAnxVI is expected to inhibit MARCKS phosphorylation in wildtype cells. In contrast, ψAnxVI peptide will promote MARCKS phosphorylation in wildtype cells, but not in knockout cells. The cAnxVI is not expected to affect the outcome of the assay.

The role of α-PKC in cancer can be determined. The α-PKC has been shown to play a role in both tumor growth and angiogenesis. Human prostate cancer mouse model showed that inhibition of α-PKC blocks tumor growth. Peptides described above will be administered in a continuous fashion through an implanted pump, and tumor size will be assayed at different time points of progression. In addition, angiogenesis will be measured at the same time points. It is expected that pAnxVI will slow the tumor growth, and potentially decrease angiogenesis, while ψAnxVI will increase tumor growth rate and increase angiogenesis. The cAnxVI peptide should not affect the outcome of the study.

While a number of exemplary aspects and embodiments have been discussed above, those of skill in the art will recognize certain modifications, permutations, additions and sub-combinations thereof. Further, one of skill will appreciate that the teachings provided above illustrate general concepts, and several variations are contemplated. For example, where listings of options are provided, for example in a Markush group, one of skill will appreciate that one or more components of those listings may be removed as unsuitable or undesirable for use in some embodiments and/or may be useful in other embodiments. It is intended that the following appended claims and claims hereafter introduced are interpreted to include all such modifications, permutations, additions and sub-combinations as are within their true spirit and scope.

It is claimed:

1. A peptide comprising the amino acid sequence J_{N}-DXₐₐ₁DXₐₐ₂LI-J_{C} (SEQ ID NO:1), or a conservatively modified variant thereof, wherein
Xₐₐ₁ is selected from a group consisting of E, R, A, and conservative substitutions thereof;
Xₐₐ₂ is selected from a group consisting of T, V, and conservative substitutions thereof;
J_{N} comprises a component selected from a group consisting of hydrogen; poly(ethylene glycol); methyl; T, GT, LGT, I, HI, AHI, and conservative substitutions thereof; and combinations thereof; and,
J_{C} comprises a component selected from a group consisting of hydroxyl; poly(ethylene glycol); amino; and E, EI, EIL, V, VL, VLV, and conservative substitutions thereof; and combinations thereof.

2. The peptide of claim 1 comprising DEDTLI (SEQ ID NO:2) or a conservatively modified variant thereof.

3. The peptide of claim 1 comprising DRDVLI (SEQ ID NO:3) or a conservatively modified variant thereof.

4. The peptide of claim 1 further conjugated to a carrier peptide.

5. The peptide of claim 4, wherein the carrier peptide comprises TAT (SEQ ID NO:4), Drosophila Antennapedia homeodomain (SEQ ID NO:5), or a polyarginine.

6. A peptide comprising the amino acid sequence J_{N}-VAXₐₐ₁Xₐₐ₂Xₐₐ₃LXₐₐ₄-J_{C} (SEQ ID NO:6), or a conservatively modified variant thereof, wherein
Xₐₐ₁ is selected from a group consisting of R, D, A, and conservative substitutions thereof;
Xₐₐ₂ is selected from a group consisting of V, E, and conservative substitutions thereof;
Xₐₐ₃ is selected from a group consisting of E, K, A, and conservative substitutions thereof;
Xₐₐ₄ is selected from a group consisting of K, H, and conservative substitutions thereof;
J_{N} comprises a component selected from a group consisting of hydrogen; poly(ethylene glycol); methyl; A, SA, LSA, E, AE, KAE, and conservative substitutions thereof; and combinations thereof; and,
J_{C} comprises a component selected from a group consisting of hydroxyl; poly(ethylene glycol); amino; and G, GT, GTV, V, VT, VTV, and conservative substitutions thereof; and combinations thereof.

7. The peptide of claim 6 comprising VARVELK (SEQ ID NO:7) or a conservatively modified variant thereof.

8. The peptide of claim 6 comprising VADEKLH (SEQ ID NO:8) or a conservatively modified variant thereof.

9. The peptide of claim 6 further conjugated to a carrier peptide.

10. The peptide of claim 9, wherein the carrier peptide comprises TAT (SEQ ID NO:4), Drosophila Antennapedia homeodomain (SEQ ID NO:5), or a polyarginine.

11. A peptide comprising the amino acid sequence J_{N}-QXₐₐ₁EXₐₐ₂Xₐₐ₃A-J_{C} (SEQ ID NO:9), or a conservatively modified variant thereof, wherein
Xₐₐ₁ is selected from a group consisting of V, A, and conservative substitutions thereof;
Xₐₐ₂ is selected from a group consisting of Q, D, A, and conservative substitutions thereof;
Xₐₐ₃ is selected from a group consisting of D, E, and conservative substitutions thereof;
J_{N} comprises a component selected from a group consisting of hydrogen; poly(ethylene glycol); methyl; A, EA, DEA, L, SL, GSL, and conservative substitutions thereof; and combinations thereof; and,
J_{C} comprises a component selected from a group consisting of hydroxyl; poly(ethylene glycol); amino; and Q, QA, QAL, N, NQ, NQP, and conservative substitutions thereof; and combinations thereof.

12. The peptide of claim 11 comprising QVEQDA (SEQ ID NO:10) or a conservatively modified variant thereof.

13. The peptide of claim 11 comprising QAEDEA (SEQ ID NO:11) or a conservatively modified variant thereof.

14. The peptide of claim 11 further conjugated to a carrier peptide.

15. The peptide of claim 14, wherein the carrier peptide comprises TAT (SEQ ID NO:4), Drosophila Antennapedia homeodomain (SEQ ID NO:5), or a polyarginine.

16. A method of decreasing a cellular response to β-protein kinase C (β-PKC) (βI is SEQ ID NO:12; βII is SEQ ID NO:34), wherein the method comprises
adding the peptide of claim 2 to an environment comprising a cell, annexin I (SEQ ID NO:13), and β-PKC; wherein,
the peptide binds to the β-PKC and inhibits binding between the β-PKC and the annexin I.

17. The method of claim 16, wherein the cellular response includes translocation of the β-PKC from the cytosolic fraction of cell to the particulate fraction of the cell.

18. The method of claim 16, wherein the cellular response includes cellular hypertrophy.

19. The method of claim 16, wherein the cell response includes hyperproliferation.

20. A method of increasing a cellular response to β-protein kinase C (β-PKC) (βI is SEQ ID NO:12; βII is SEQ ID NO:34), wherein the method comprises:
adding the peptide of claim 3 to an environment comprising a cell, annexin I (SEQ ID NO:13), and β-PKC; wherein,
the peptide binds to the β-PKC and promotes binding between the β-PKC and the annexin I.

21. The method of claim 20, wherein the cellular response includes translocation of the β-PKC from the cytosolic fraction of cell to the particulate fraction of the cell.

22. The method of claim 20, wherein the cellular response includes cellular hypertrophy.

23. A method of decreasing a cellular response to α-protein kinase C (α-PKC) (SEQ ID NO:14), wherein the method comprises
adding the peptide of claim 7 to an environment comprising a cell, annexin VI (SEQ ID NO:15), and α-PKC; wherein,
the peptide binds to the α-PKC and inhibits binding between the α-PKC and the annexin VI.

24. The method of claim 23, wherein the cellular response includes translocation of the α-PKC from the cytosolic fraction of cell to the particulate fraction of the cell.

25. The method of claim 23, wherein the cellular response includes hyperproliferation.

26. A method of increasing a cellular response to α-protein kinase C (α-PKC) (SEQ ID NO:14), wherein the method comprises:
adding the peptide of claim 8 to an environment comprising a cell, annexin VI (SEQ ID NO:15), and α-PKC; wherein,
the peptide binds to the α-PKC and promotes binding between the α-PKC and the annexin VI.

27. The method of claim 26, wherein the cellular response includes translocation of the α-PKC from the cytosolic fraction of cell to the particulate fraction of the cell.

28. The method of claim 26, wherein the cell response includes hyperproliferation.

29. A method of decreasing a cellular response to δ-protein kinase C (δ-PKC) (SEQ ID NO:16), wherein the method comprises:
adding the peptide of claim 12 to an environment comprising a cell, annexin V (SEQ ID NO:17), and δ-PKC; wherein,
the peptide binds to the δ-PKC and inhibits binding between the δ-PKC and the annexin V.

30. The method of claim 29, wherein the cellular response includes translocation of the δ-PKC from the cytosolic fraction of cell to the particulate fraction of the cell.

31. The method of claim 29, wherein the cellular response includes reperfusion following ischemia.

32. A method of increasing a cellular response to δ-protein kinase C (δ-PKC) (SEQ ID NO:16), wherein the method comprises:
adding the peptide of claim 13 to an environment comprising a cell, annexin V (SEQ ID NO:17), and δ-PKC; wherein,
the peptide binds to the δ-PKC and promotes binding between the δ-PKC and the annexin V.

33. The method of claim 32, wherein the cellular response includes translocation of the δ-PKC from the cytosolic fraction of cell to the particulate fraction of the cell.

34. The method of claim 32, wherein the cellular response includes reperfusion following ischemia.
